(19) United States
(12) Patent Application Publication
(10) Pub. No.: US 2012/0204865 A1
(43) Pub. Date: Aug. 16, 2012

(54) SYSTEMS AND TECHNIQUES FOR PERFORMING GASTROINTESTINAL PROCEDURES

(76) Inventors: Charles J. Filipi, Omaha, NE (US); Timothy B. Hunt, Miami Beach, FL (US)

(21) Appl. No.: 13/379,830

(22) PCT Filed: Jun. 24, 2010
(86) PCT No.: PCT/US2010/039879

§ 371 Date: Mar. 19, 2012

Related U.S. Application Data
(60) Provisional application No. 61/220,089, filed on Jun. 24, 2009.
(60) Provisional application No. 61/221,545, filed on Jun. 29, 2009.

(51) Int. Cl. A61M 16/01; A61B 17/32; A61B 17/04

(52) U.S. Cl. 128/200.26; 606/144; 606/170

(57) ABSTRACT

A system for performing minimally invasive gastrointestinal procedures includes an excision device 100 and a suturing device 200. The excision device 100 has a proximally tapered suction cavity 131 with extendable wires 160 to prevent capture of unwanted tissue. The suturing device 200 uses a squeeze trigger 202 to drive an array of circular needles 232.

### RELATED APPLICATION DATA

This application claims the benefit of U.S. Provisional application No. 61/220,089 filed Jun. 24, 2009, which is incorporated by reference. This application also claims the benefit of U.S. Provisional application No. 61/221,545 filed Jun. 29, 2009, which is also incorporated by reference.

### BACKGROUND

The present invention generally relates to equipment and procedures for use in performing minimally invasive procedures. More specifically, but not exclusively, the invention relates to equipment for minimally invasive procedures which are performed on the gastrointestinal tract and/or which involve either the resection of tissue or the application of sutures to tissue or both.

There is considerable interest in performing surgical procedures in the gastrointestinal tract in a minimally invasive manner. Particular areas of interest are procedures which may effectively perform tissue modifications and repairs near the esophagus, such as in treating gastro esophageal reflex disease (GERD), or in the stomach, such as in performing a gastroplasty or other type of gastric reduction surgery. However, these are particularly challenging areas to work in and there are needs for additional tools and procedures.

### SUMMARY

The present invention provides systems and techniques for use in performing gastrointestinal procedures. While the actual nature of the invention covered herein can only be determined with reference to the claims appended hereto, certain aspects of the invention that are characteristic of the embodiments disclosed herein are described briefly as follows.

In one form, a system for performing a gastrointestinal procedure comprises a standalone excision device having a side disposed suction cavity and a standalone transverse suture application device having a side disposed suction cavity, wherein at least one dimension of the respective suction cavities are different. For example, transverse sutures might be applied across a uniform suction cavity whereas a proximally tapered suction cavity may be used to form a flared excision bed.

In another form, an improved tissue excision device is provided wherein extendable members are provided along the lateral sides of a suction cavity and extend outwardly upon activation by an extender so as to prevent the capture of unwanted tissue in the first suction cavity.

In another form, an improved tissue excision device is provided wherein a platform is provided in the floor of the suction cavity and is raisable upon activation by a platform actuator so as to facilitate removal of the excised tissue.

In another form, an improved transverse suturing device is provided wherein circular needles are contained in channels defined between adjacent parts of a multi-part capsule wherein the capsule parts can be readily separated to release the needles in case of an emergency or for purposes of reloading. For example, the parts may be biased to be apart and are held together by a compressive force which may be released at the handle.

In another form, an improved transverse suturing device is provided wherein a squeeze trigger is used to drive needles in a circular path.

In another form, an improved transverse suturing device is provided wherein a pull wire actuated drive shuttle engages the interior side of circular needles

These and other aspects are described more fully below.

### BRIEF DESCRIPTION OF THE FIGURES

Although the characteristic features of this invention will be particularly pointed out in the claims, the invention itself, and the manner in which it may be made and used, may be better understood by referring to the following description taken in connection with the accompanying figures forming a part thereof.

FIG. 1 is a perspective view of a kit comprising an excision device, a suturing device, and a knotter.

FIG. 2 is a perspective view of an excision device.

FIG. 3 is an enlarged underside view of the handle section of the FIG. 2 excision device.

FIG. 4 is an enlarged view of the working portion of the FIG. 2 excision device.

FIG. 5 is a side view of the working portion of the FIG. 2 excision device.

FIG. 6 is an end perspective view of the FIG. 2 excision device showing the remote tissue capture prevention wires in their extended configuration.

FIG. 7 is a perspective view of a suturing device.

FIG. 8 is an enlarged view of the distal end of the suturing device with the capsule sections extended.

FIG. 9 is an enlarged view of the proximal capsule part of FIG. 8.

FIG. 10 is an exploded view of the handle of the suture device of FIG. 7.

FIG. 11 is an exploded view of the squeeze trigger and drive mechanisms.

FIG. 12 is a perspective view of a suture device resting on a suture management accessory.

FIG. 13 is a view of an airway biteblock.

FIG. 14 is a perspective view of the biteblock of FIG. 13.

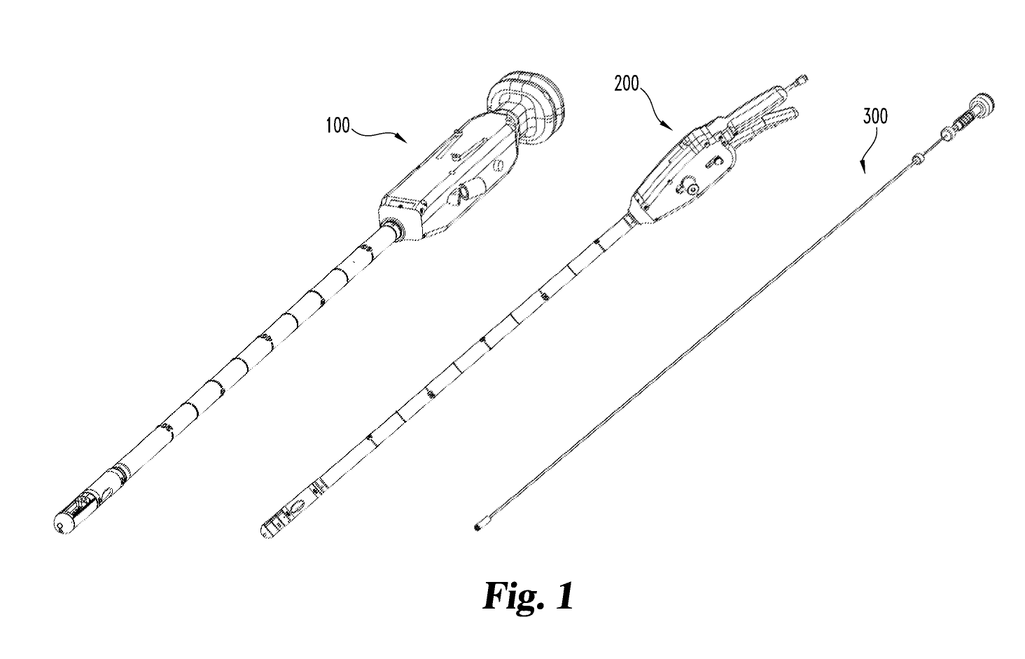

### DESCRIPTION OF EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended. Alterations and further modifications in the illustrated devices, and such further applications of the principles of the invention as illustrated herein are contemplated as would normally occur to one skilled in the art to which the invention relates.

### General Overview: Excision And Suturing

In one aspect, the present invention involves the provision of two main surgical components for a minimally invasive gastrointestinal procedure, an excision component and a suturing component. The general function of the excision component is to capture tissue from a target area in a side disposed suction cavity and then excise a layer of the captured tissue so as to form an exposed section of tissue. The excision device is made to move about the target area so as to produce multiple areas or beds of exposed tissue. The general function of the suturing component is to capture tissue from the target area in a side disposed suction cavity and to apply transverse sutures to the captured tissue. The suturing component also moves about the target area so as to apply sutures in desired locations. The applied sutures are subsequently tied together in an effort to place excised tissue beds in apposition so as to modify the shape of the target gastrointestinal area.

In the illustrated embodiment, these two surgical components are provided by two standalone devices, an excision device 100 and a suturing device 200. The excision device 100 and the suturing device 200 are each sized and configured to be introduced transorally and to operate on tissue in the stomach and/or near the gastroesphageal junction (GEJ). Each is also designed to be introduced independently over a preinserted guide wire, and each includes an endoscope lumen to allow visualization via a standard endoscope. The provision of a tailored excision device and a tailored suturing devices provide important flexibility and improved chances of positive outcomes, particularly in the context of attempts to make lasting surgical modifications in the stomach or near the gastroesophageal junction (GEJ).

Other embodiments are envisioned wherein the excision component and the suturing component could be integrated into a single device using a common suction cavity for both excision and suturing. Such an integrated device approach may have applicability in some applications, but the requirement of using common suction cavity for both suturing and excision may prove unworkable for other applications.

### Excision Device

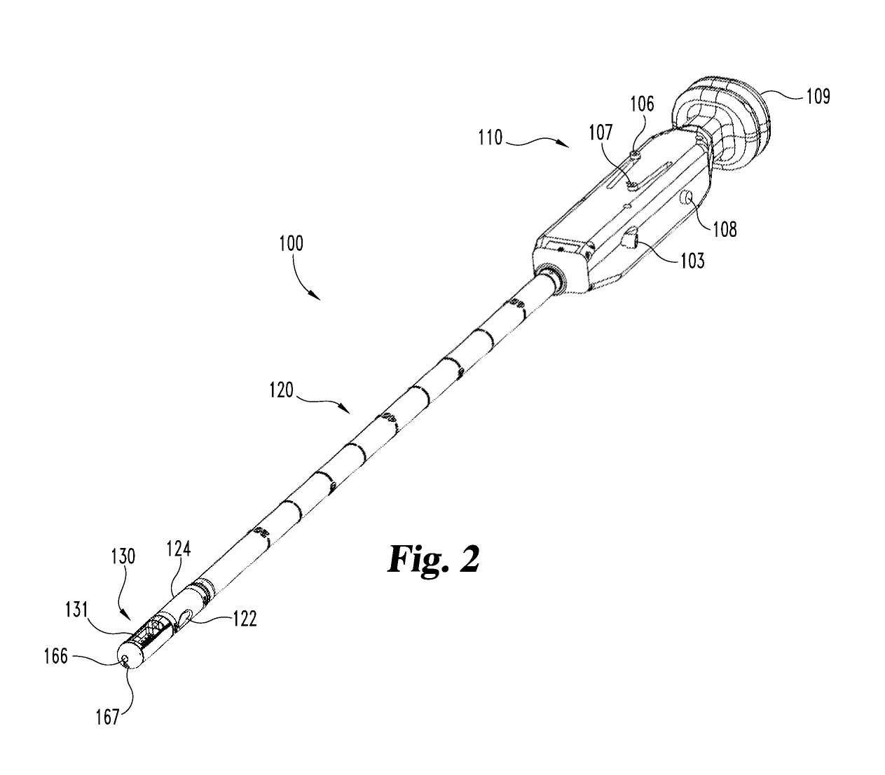

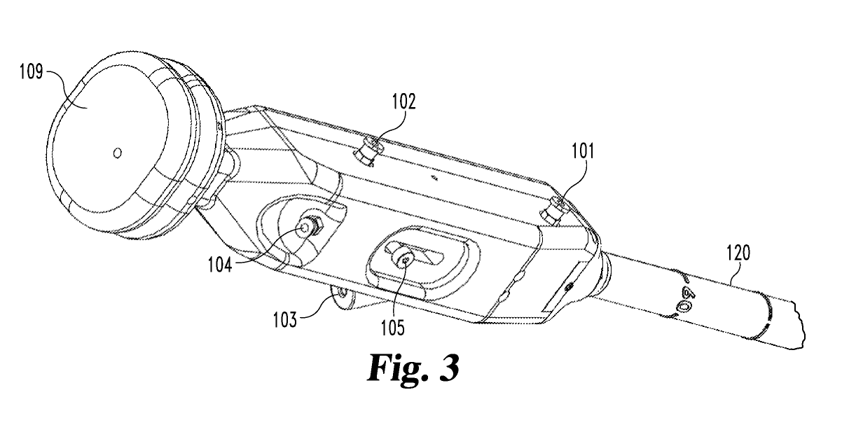

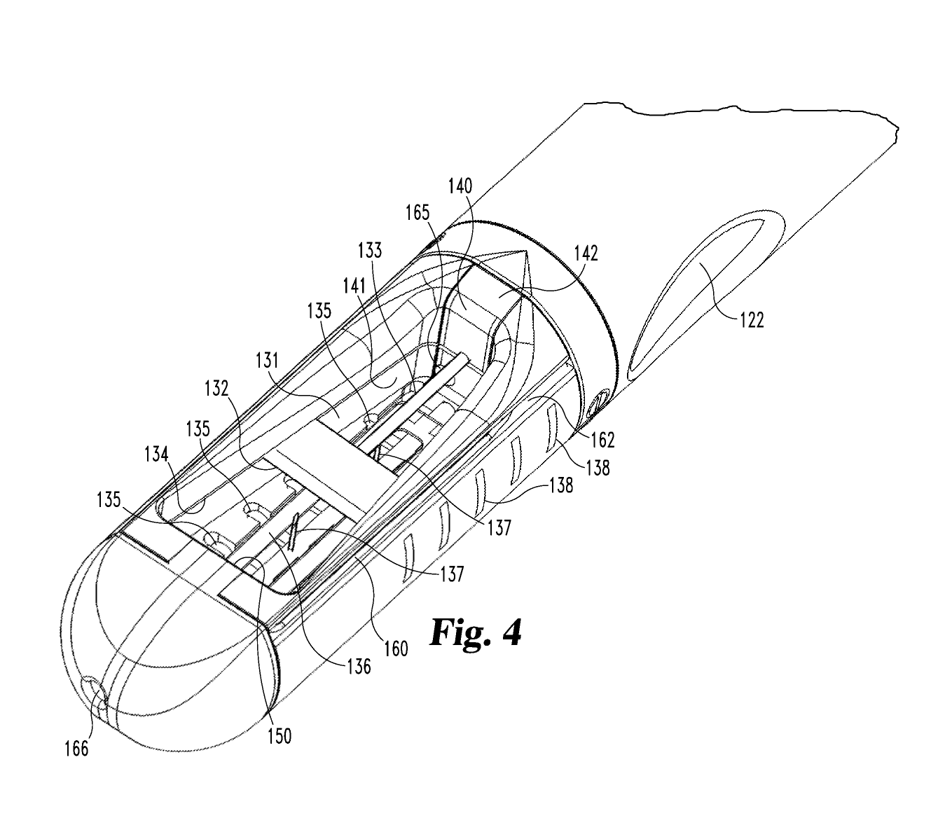

Turning now to FIGS. 2-6, excision device 100 includes a handle section 110 and a working section 130 coupled by an elongated flexible shaft 120. The excision device is designed to be inserted over a guidewire (not shown) which enters the distal tip at guidewire port 166 and, after passing through suction cavity 131, reenters the device at guidewire port 165 (FIG. 4) and exits the handle at guidewire exit port 108. The excision device is also designed to be used with a conventional 5-6.5 mm endoscope (not shown) which enters the handle at endoscope entrance port 103 and exits from the endoscope outlet port 122 in a transition section 124 between the shaft 120 and a distal capsule forming the working portion 130.

Handle section 110 includes insufflation port 102 which is designed to be connected to an external source of positive pressure via a conventional leur type fitting. The insufflation port 102 leads to an insufflation lumen (not shown) which runs the length of the device 100 to an insufflation port 167 in the distal tip, and this is used to apply positive pressure inside the patient, for example to inflate the stomach. To prevent air from leaking back through the guidewire lumen (i.e. via port 165) when positive pressure is being delivered through the insufflation port 167, the guidewire port 108 in the handle section 110 may be equipped with an valve or stopper to block the guidewire port 108 when not in use. Air leakage through the endoscope lumen may be reduced by the provision of a flexible seal at the endoscope entrance port 103.

The suction port 101 in the handle section 110 is designed to be connected to an external vacuum source via a conventional leur type fitting. Suction port 101 is connected via a suction lumen (not shown) to the suction ports 135 in the floor of the suction cavity 131 such that application of a vacuum at port 101 serves to draw tissue into cavity 131.

The injection port 104 in the handle 110 is fluidly coupled to the injection needles 137 and is used to deliver an injectate into the captured tissue prior to excision. In one mode of use, a volume of 8 cc of saline and adrenaline solution is injected into is the gastric tissue between the mucosal and muscle layers with the three fixed injection needles 137. The injection fluid swells the mucosal layer thereby pushing the junction between the mucosal and muscle layers to be in line with the excision blade which defines the depth of cut. The injection fluid may serve to prevent bleeding and perforation of serosa. Preferably, the tips of the injection needles are sized to be just under the excision blade so that the point inside the tissue where the fluid is delivered is as close as practical to where the cut occurs.

The injection needles 137 are fixed and extend upwardly from the bottom of the cavity 131 such that they penetrate the tissue as it is drawn down to the cavity floor by suction ports 135. The injection needles 137 are arranged generally along the longitudinal centerline of the suction cavity 131 so as to be generally equidistant from the opposing sidewalls 141 of the suction cavity 131, and the needles 137 are connected to a manifold to ensure equal flow of volume from each needle. The suction ports 135 are provided in the floor of the cavity 131 and are disposed on either side of the centrally located injection needles 137 such that, when suction is applied, generally equal amounts of suction are provided to the tissue on either side of the injection needles 137, which helps to assure good penetration of the injection needles 137 into the tissue. Rather than being fixed in position in the floor of the cavity 131, the injection needles could be configured so as to be selective retractable and extendable from the floor or from the sides of the cavity.

One benefit to having the injection needles 137 extending from the floor of the cavity 131 and engaged in the tissue during the excision is that the engagement between the tissue and the injection needles 137 helps to secure the tissue in place during the excision operation. In other words, the engaged tissue has increased ability to stay in place while a cutting device is passing through it (i.e. it avoids “bunching up”). Other forms of increasing the relative engagement of the tissue to the floor may also be provided, such as by adding texturing or other surface features to the floor of the suction cavity 137. Alternative or in addition, rather than having suction ports 135 that are flush with the plane of the floor, the suction ports may be tilted towards the direction of the advancing cutting device.

After excision, the tissue which has been captured in cavity 131 is designed to be excised by an appropriate tissue cutter, such as a wire or a blade. To assure that the excision device is not activated prematurely, a lockout mechanism (not shown) may be provided to prevent actuation of the tissue cutter. The lockout mechanism may be designed to be released automatically once fluid is injected or a device is coupled to the injection port.

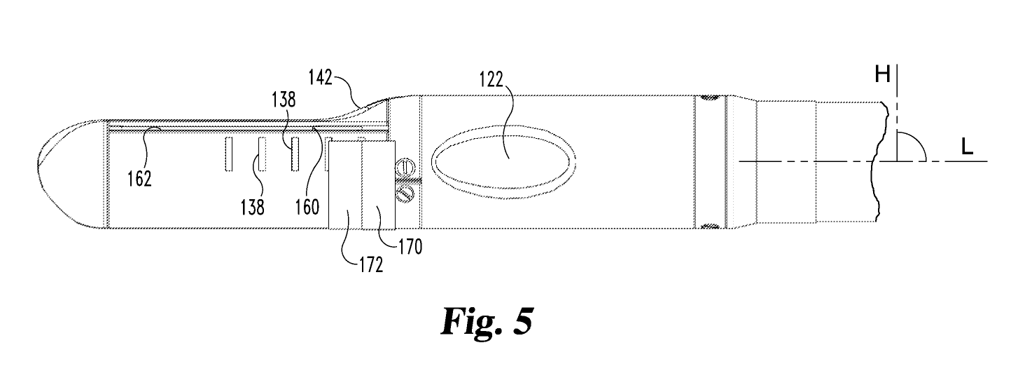

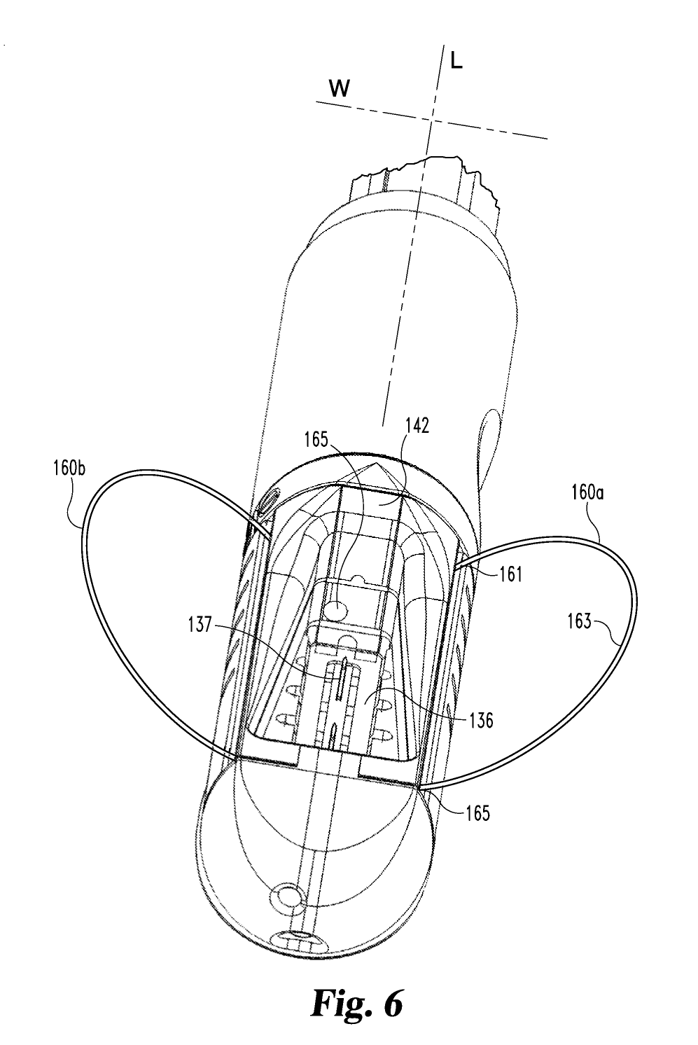

In the illustrated embodiment, a blade 132 is positioned in guiding slots 134 in opposing side walls 141 of the suction cavity 131. The guiding slots 134 run parallel to the floor of the suction cavity 131 so as to provide a relatively uniform depth of excision across the longitudinal length of the cavity 131, wherein it is to be understood that “length” refers to axis L, “height” and “depth” refer to axis H, and “width” refers to axis W as indicated in FIGS. 5 and 6. Providing a uniform excision depth is useful to assure that only a surface layer of tissue is excised.

Applicant has found, however, that the width of the suction cavity 131 need not be uniform across its longitudinal length and that providing variation in the width of the suction can be particularly advantageous in a number of application. For example, in the illustrated embodiment, the cavity 131 is substantially wider at its distal end 150 than at its proximal end 140 with the width steadily increasing as you proceed distally. Because the width of the cavity 131 is flared distally, the width of the exposed tissue resulting from the excision will likewise be flared distally.

The formation of exposed tissue sections that are flared distally can have a number of useful applications, and applicant has found this geometry to be particularly useful when excising stomach tissue near or adjacent to the gastroesophageal junction (GEJ). This is because the volume of the stomach expands as you proceed into the stomach from the GEJ and thus the effective surface area of stomach tissue at a certain distance from the GEJ increases with distance from GEJ (i.e. similar to an inverted funnel).

In addition to having a width that is narrower, the proximal end 140 of the cavity 131 also differs from the distal end 150 due to the presence of shoulder section 142. As shown in FIG. 5, shoulder section 142 is an elevated tapered section adjacent the proximal end 140 which serves to smooth the transition to the shaft 120 and also serves to restrict extra tissue from sliding or otherwise being drawn into the distal end 150 of cavity 131. The combination of the proximally narrowed width of the suction cavity and the proximal shoulder section 142 can be an effective way to reduce the risk of perforation when the device is used to excise tissue near the proximal GEJ.

To further reduce the risk of such perforations, the working portion 130 of excision device 100 may be provided with visual marking designed to assure accurate placement of the device 100. For example, laser etched distance markings 138 may be provided along the side of the working portion 130 and color coded position indicating stickers 172, 170 may be affixed to the side of the working portion to visually identify the desired location on the working portion 130 that should be positioned at a particular location in the anatomy. For example, based on a visual evaluation of the GEJ with the endoscope, the operator may determine where the proximal shoulder section 142 should be located and then how much of the working portion 130 would be visible inside the stomach when the shoulder section 142 is in the desired position. The different colored stickers 172, 170 might then be placed on the working portion 130 on either side of the indicated location. Then, by watching for the stickers to become visible to an endoscope inside the stomach, the operator can determined when the shoulder section 142 is placed in the desired position.

Excision device 200 is further provided with wires 160a and 160b which are individually extendable upon activation by actuators 107 and 106 respectively. In their unextended configuration (FIG. 4), the wires 160a, 160b are disposed in recesses which extend along the lateral sides of the cavity 131. In this position, the outer surface of the wires 160a, 160b are generally flush with or recessed below the adjacent outer surface of the device so as to preserve a generally smooth outer surface profile for device 100.

The wires 160a, 160b have their distal ends 165 crimped or otherwise relatively fixed in position near the distal end 150 of cavity and their proximal ends coupled to the actuators 107, 106 in the handle. The wires 160a, 160b may be made of a shape memory and/or superelastic material, such as Nitinol, such that sliding actuator 107 distally provides additional wire into the proximal portion of the channel and causes wire 160a to bow radially outwardly into an arc shape as shown in FIG. 6, wherein the arced wires 160a, 160b are generally disposed in a plane which is at an acute angle (e.g. about 45°) from horizontal.

In the extended configuration of FIG. 6, a proximal section 161 of the wire is near the proximal end of the cavity 131, a distal section 165 of the wire is near the distal end 150 of the cavity, and a midsection 163 of the wire is spaced from the outer surface of the device 100 by an amount which depends on the degree of actuation and the shape of the extended wire. For example, if the extended wire were in the form of a half-circle, the midsection 163 would be spaced one radius from the outer surface, which would correspond to about ½ the distance between sections 165 and 161, or approximately ½ the length of the suction cavity 131. A variety of different extended wire shapes and configurations may be employed.

Movement of the wire 160a, 160b from the unextended (FIG. 4) to extended (FIG. 6) positions may be accomplished mechanically, for example, by releasing tension applied to keep it in its retracted position or applying compression to force it to transition outward. Movement may also be accomplished electrically, for example, by applying current to the wire.

The wires may generally be used in a number of different ways. For example, one or more of the extended wires can be used as a tissue manipulator by bringing it into contact with tissue and then rotating the excision device 100 about its longitudinal axis and/or by moving the device 100 proximally or distally. In another use, the wires are extended in conjunction with application of suction to suction cavity 131 and serve to prevent unwanted tissue from being captured in the suction cavity 131. In still other forms, the extended wires apply pressure to either side of the suction trough (i.e. push the trough back) to enable only the tissue in line with the suction trough to be influenced by suction.

Another aspect of the excision device 100 is the provision of a raisable platform 136 in the floor of the suction cavity 115. As shown in FIG. 6, platform 136 is provided in a central cutout in the center of the cavity and runs along either side of the injection needles 137. The underside of platform 136 is connected to wires (not shown) which are actuated by sliding actuator 105 on handle. Pushing actuator 105 distally forces the platform wires up and thus the platform 136 is raised up from its position in the cavity floor. This may be used to assist in removing cut tissue from the cavity, particularly in the case where the tissue may have become stuck or is otherwise difficult to dislodge.

### Suturing Device

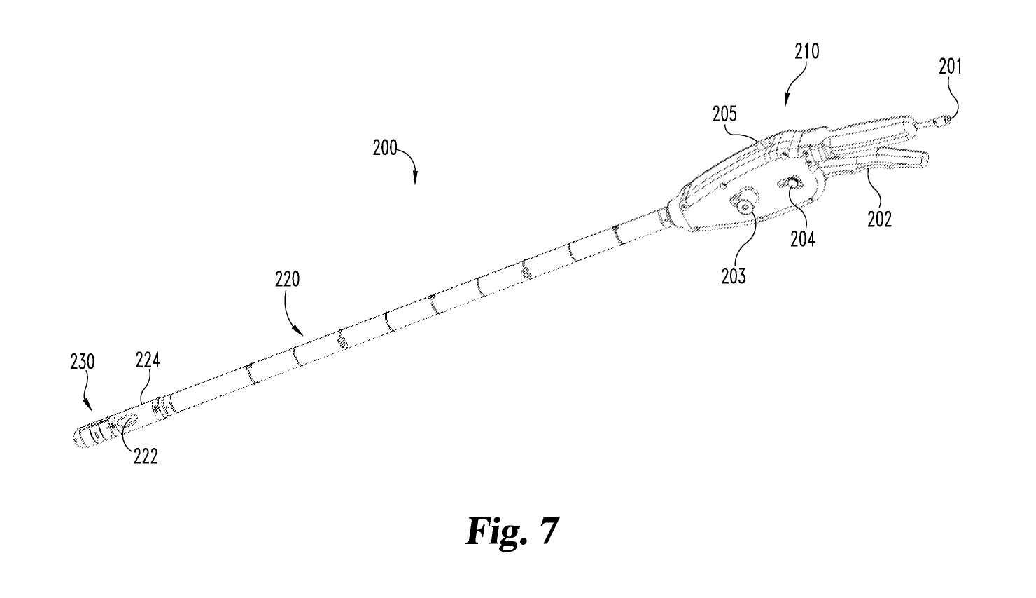

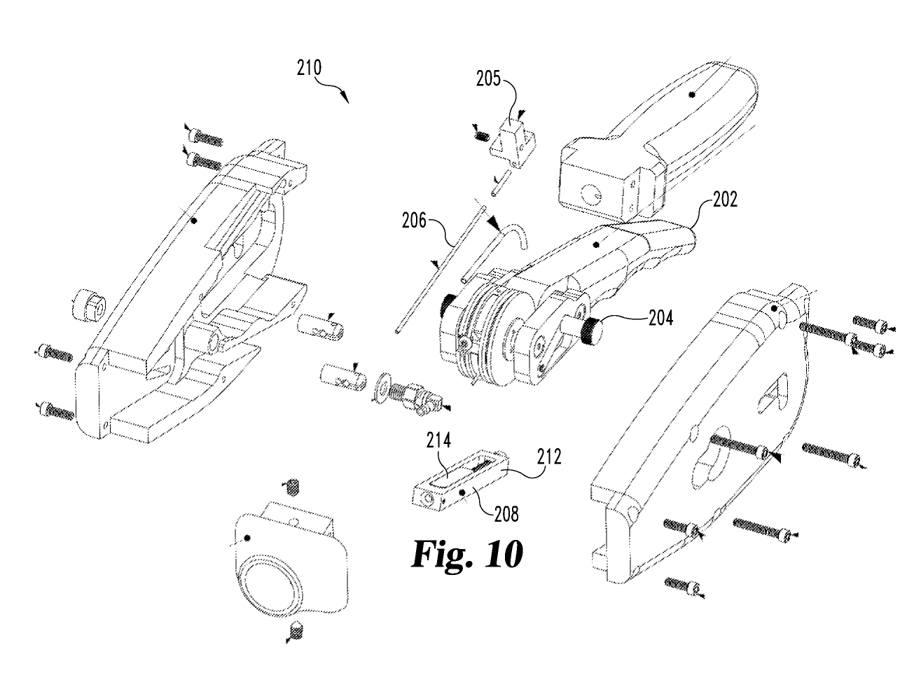

Turning now to FIGS. 7-11 the suturing device 200 is configured similar to the resection device 100 and includes an elongated shaft 220 connecting a 210 handle section with a distal working portion 230. The suturing device 200 is designed to be inserted over a standard guide wire via a guide wire lumen that extends from an entrance port (not shown) in the handle, through the suction cavity via hole 265 and eventually out exit hole 266 in the distal tip. An endoscope can be inserted via entrance port 201 in the handle 210 and exits the device 200 via outlet hole 222 in transition section 224.

The working portion 230 includes a side disposed suction cavity 231 and an array of needles 232 which are designed to apply transverse sutures to tissue captured in the suction cavity. Tissue is captured when vacuum is applied to ports 221 via lumen 275 which extends to a suction port (not shown) in handle section. In one contemplated application the suturing device 200 is designed such that it applies full thickness sutures through gastric tissue while avoiding suturing tissue that is external to serosa of the target gastric tissue, and selection of a suction cavity which is 30 mm long and 8 mm wide can facilitate capturing the proper quantity of gastric tissue.

The working portion 230 is composed of three capsule parts 240, 241, 242 which are maintained in alignment via a pair of alignment pins. More specifically, alignment pin 237 extends through the alignment lumens 274 of all three capsule parts, and a corresponding alignment pin (not shown) extends through lumens 273.

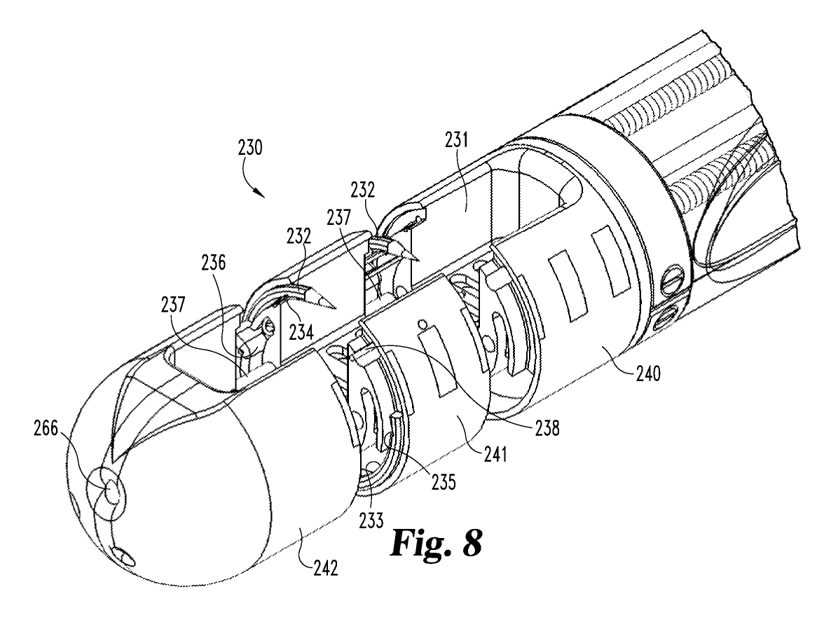

The three capsule parts are held together via application of a compressive force which is maintained by a tension controller 208 mounted in the handle. More specifically, one end of a tension cord (not shown) is secured to the distal capsule part 242 and the tension cord extends proximally through the tension lumens 270 of the middle 241 and proximal 240 parts, through the shaft 220 and up to the tension controller 208 in the handle 210. The tension lumens 270 are designed with a recessed lip that engages with a compression spring (not shown) provided over the tension cord and disposed between each pair of adjacent capsule parts. The presence of the compression spring between adjacent parts biases the capsule parts to spread apart (FIG. 8) when the compressive force is sufficiently released.

The tension controller 208 is designed to provide fine levels of tension adjustments, such as to compensate for articulation of the shaft, and to provide for quick or emergency release of tension so as to cause the three capsule parts to spread apart. For example, tension controller 208 may be in the form of an external frame 212 and a tension wire mount 214 whose position within the frame 212 is subject to fine adjustments, for example by turning a threaded rod. The external frame 212 may then be mounted to a fixed location in the handle but in a quick release format.

The ability to do a quick release of tension is a significant safety advantage, particularly considering that the needles are positioned in the device so as to be the external most component once the capsules parts are spread apart. For example, if an emergency arose while the suture device was midway through a suturing cycle, the needles would still be engaged in the tissue and the suturing device would be effectively sewn into the patient. Without a way to quickly release the needles, the doctor would need to complete the suturing cycle before the device could be removed. The ability to do a quick release presents a new option. Now if an emergency arises, the doctor can perform a quick release of tension and abandon the suture needles wherever they happen to be, with the understanding that it may be better for the patient to endure a subsequent procedure to retrieve something left behind than bear the immediate risk of the emergency.

Returning now to the actuation of the needles, each needle 232 is circular and has three notches on its interior surface 233 with each notch spaced 180° apart. As shown in FIG. 8 notch 234 is near the tip, notch 235 is near the tail, and a third notch (not shown) is generally at the midsection of the needle. Suture thread (not shown) is also preferably coupled to each needle near its midsection with the free end of the thread from each needle being retuned through its own suture return lumen 272 or 271 which are accessed via a suture inlet hole in the distal tip.

The needles 232 and their associated drive shuttles 236 are mounted in the channels 253, 263, 262 in the capsule parts. Needles 232 are positioned in the outer circular channels 263, 262 and the drive shuttle 236 is disposed to the interior side of the needle 232 in the lower channel 253. Drive shuttle 236 includes a one way spring latch (not shown) which engages one of the interior notches (e.g. 234, 235) for purposes of driving the needle, and the shuttle 236 has an internal channel which extends around the sides of the needle 232 for purposes of supporting and assuring proper alignment.

The shuttle is coupled to first and second pull wires (not shown) which extend distally through lumens 278 and 279 respectively. The shuttle is constrained by the presence of upper sidewall 250 to arc back and forth in hemispherical channel 253 from one end 251 to the other 252 based on whether greater tension is being applied on the forward (279) or backward (278) pull wire. If there is no tension differential (i.e. the wires are in balance) the shuttle 236 would stand still.

The handle section 210 is configured such the position of the trigger 202 determines the position of the shuttle, and more specifically that squeezing the trigger 202 moves the shuttle forward towards end 252 and releasing the trigger moves the shuttle back to end 251. This may be accomplished by wrapping the pull wires in opposite directions and mounting them to hubs 310, 320 that are made to turn in the same direction. Accordingly, because the different pull wires are wrapped in opposition, rotating the hubs 310, 320 in one direction wraps/tightens one pull wire while unwrapping/loosing the other.

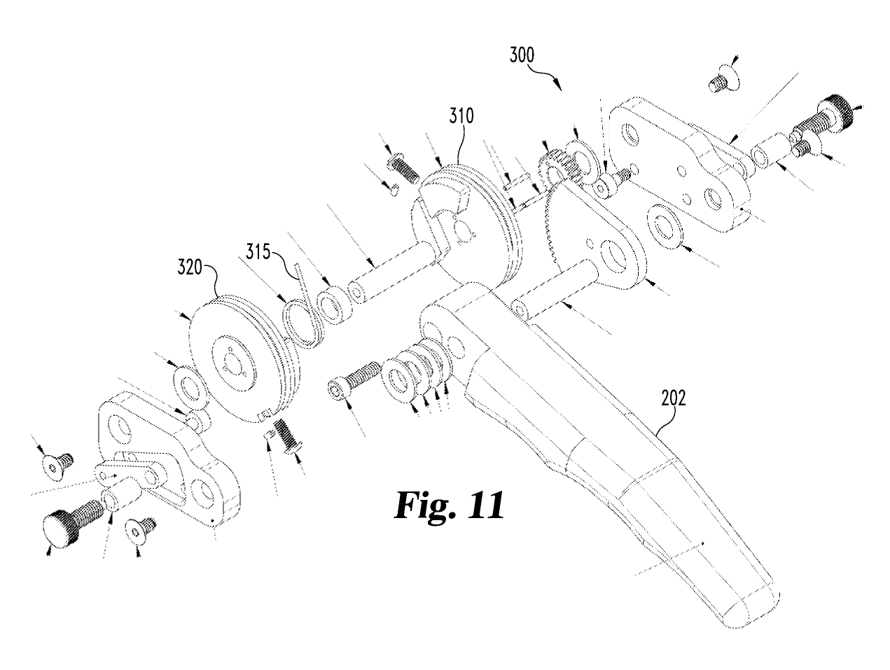

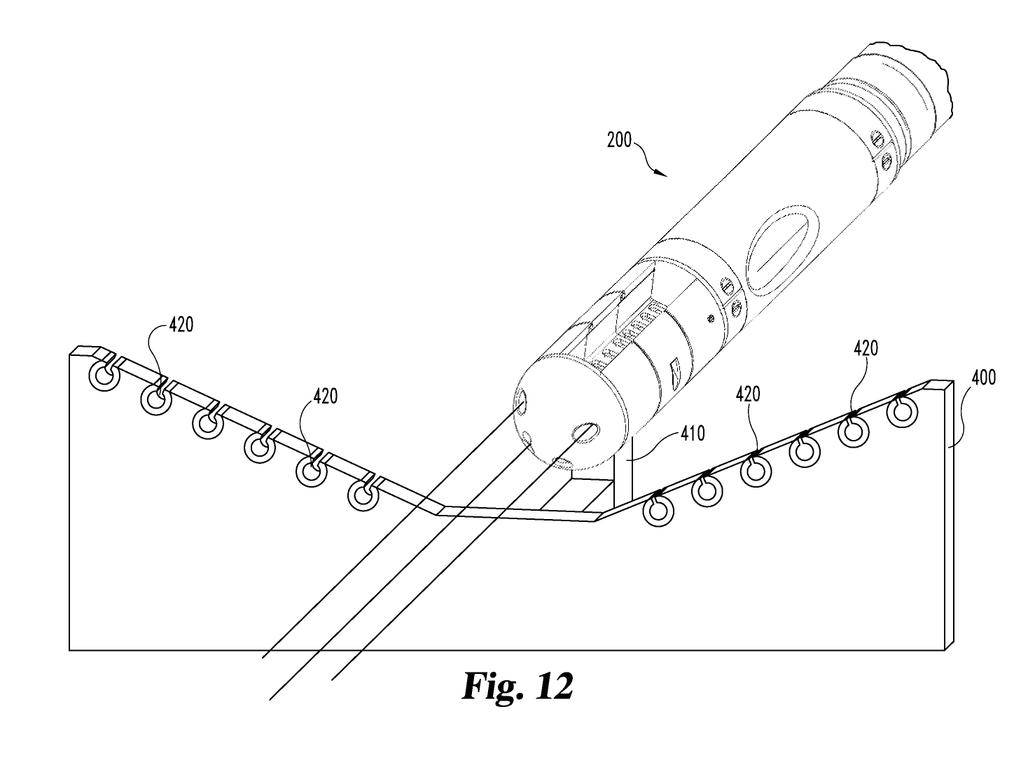

As shown in FIG. 11, hubs 310, 320 do generally turn in the same direction, but they are not rigidly coupled at all times. Rather, a torsion spring 315 is interposed between the hubs 310, 320 so as to provide a counter rotational bias that serves to assure that the pull wires are always under some level of tension. Keeping the pull wires in constant tension smoothes overall motion by taking up any unnecessary slack that might develop, for example, due to the flexing of the shaft.

Trigger 102 is pivotally mounted in the handle 210 with a slide plate pin 204 disposed in an L shaped slot so as to provide a quick release capability for releasing tension on the pull wires. During normal operation, the pin 204 is proximal and the pull wires are under tension. To maintain the state of tension, the pin 204 would typically be slid downwardly into downwardly extending section of the L shaped slot. To release tension, for example in case of an emergency, pin 204 is raised upwardly, and once clear of the corner, would be free to slide proximally to release the pull wire tension.

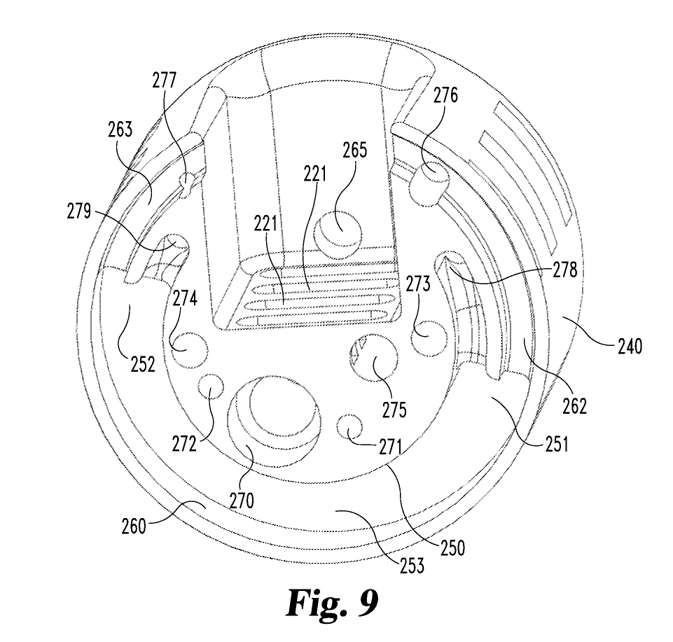

The internal notches (235, 234) on the needles 232 serve multiple purposes. In addition to serving as the engagement points for the drive shuttle 236, a spring bar 238 is provided in cavity 262 at the point (276 in FIG. 9) where the internal notch 235 at the tail end of needle will be at the completion of a full stroke. Spring bar 238 catches the tail notch 235 and holds it in this position. This is an important position because it is the only point in cycle when the needle is completely clear of the suction cavity.

As further means of securing the needle in this position, a confirmation wire 206 is extended through lumen 277 by activation of slider 205 in handle 210. With the confirmation wire 206 in position, there is even greater security and resistance to accidental needle movement. Furthermore, unlike the spring bar 238 which is always in position, the confirmation wire 206 need not be flexible and it can be sized such that, when in place, the needles are effectively locked in position. Accordingly, confirmation wire 206 can serve as a lock bar.

### Knotting/Suture Management

Once sutures have been applied, they are collected and organized outside the patient such that appropriate ones may be tied together depending on the particular procedure of interest. Rather than actually tying the together, a knotting device 300 is typically employed. As is known in the art, the knotting device would apply a crimp or sleeve or similar mechanical fastener to the desired suture wires and while pulling them tight. Knotter 300 is an example of a knotting device that might be used alongside a 10 mm endoscope.

To facilitate the suture organizational process, a suture management accessory 400 may also be provided. Suture management accessory has a central mount 410 for receiving the distal working portion of the suture device 200 and a plurality of suture holders 420 spread out to either side. As illustrated, suture holders 420 are formed in an upwardly extending fashion, which serves to reduce the chance of accidentally dislodging the centrally located ones while reaching towards the outside.

### Airway Bite Block

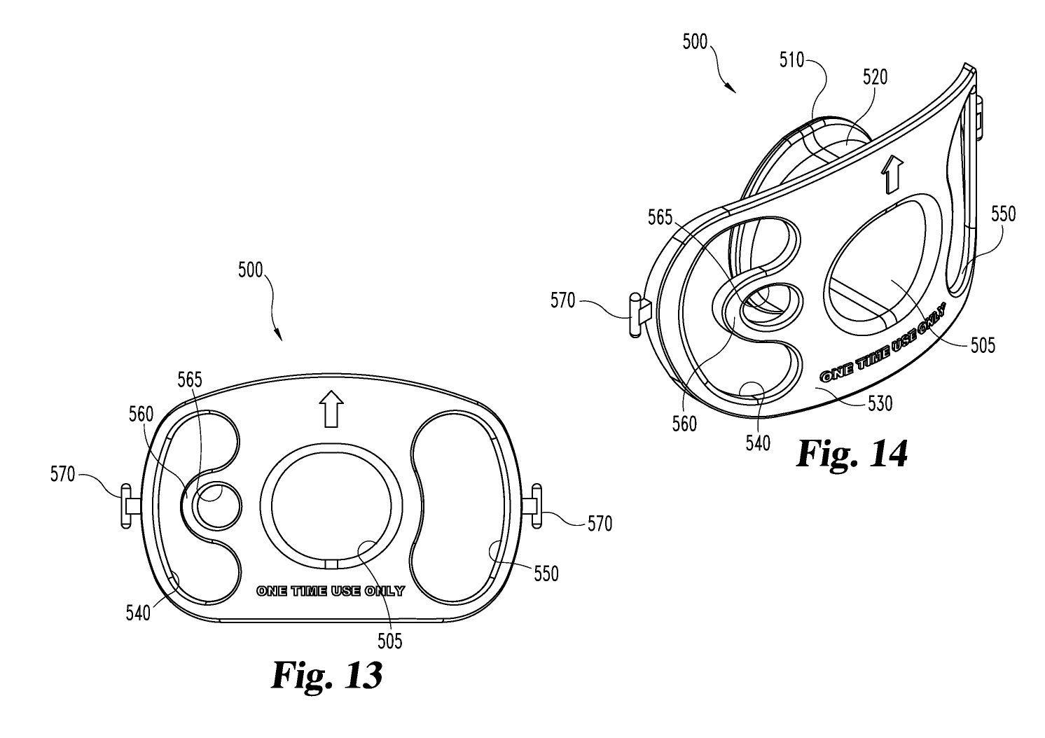

FIGS. 13 and 14 illustrate a bite block for use during the suturing and excision procedures described herein. The bite block is adapted for use with a separately inserted airway. Bite block 500 includes a main lumen 520 adapted to be positioned between the patient's teeth and to provide instrument access to the oral cavity. The bite block 500 includes a pair of side holes 540, 550 on either side of the main lumen 505, and T connectors for attaching to a head strap. An airway mount 560 is disposed in one of the side holes 540. The airway mount is molded integrally with the bite block. The purpose of the airway mount 560 is to secure an airway to the bite block such that the airway extends from one of the side holes 540 distally into the oral cavity so as to effectively provide a positive airway during a procedure.

The airway may be a flexible tube having sufficient diameter, length, and rigidity to positively maintain an airway. In one form, the airway mount is designed to accommodate a nasopharangeal airway (NPA), which are commercially available airways designed to be used nasally. Nasopharangeal airways have the shape of an elongated flexible tube with a flared, or trumpet, end. In their conventional use, the tube is inserted into a patient's nasal passage with the flared end abutting against the nostril and serving to prevent the NPA from going into the nasal cavity. Surprisingly, applicants have found that NPAs work well when inserted into the oral cavity so as to function as an oral pharangeal airway.

Airway mount 560 is specifically designed and configured to form a friction fit with a standard NPA so as to reduce the chance that the NPA would be extruded by the patient. Airway mount 560 is a round hole having an inner diameter in the range of 9-15 mm, 5-12 mm, 5-15 mm, 6-14 mm, 7-13 mm, 8-12 mm or 8-11 mm, or about 9 mm.

With respect to bite block, what has been described is the combination of a bite block and an airway, wherein the bite block comprises a body defining a main lumen which terminates distally in the oral cavity and a pair of side openings, and wherein the airway is mounted in one of the side openings and extends into the patient's oral cavity substantially past the distal edge of the main lumen. The airway may comprise an elongated flexible tube having a flared proximal end, such as nasopharyngeal airway. The airway may be secured to the bite block.via a snap fit or a friction fit mounting arrangement.

What has also been described is a system comprising a main bite block body defining upper and lower outer surfaces adapted to be positioned between a patient's upper and lower teeth respectively and defining an operating lumen for providing instrument access; wherein the bite block includes an outer face adapted to be positioned outside the patient's mouth during use, the outer face defining a pair of side openings; and a nasopharyngeal airway positioned in one of the side openings and extending distally into the patient's oral cavity so as to function as an oral pharyngeal airway. The nasopharyngeal airway may be secured to the bite block. The nasopharyngeal airway may be secured to the bite block via a strap or a mount. The nasopharyngeal airway may be secured to the bite block via a mount which is disposed within the respective side opening of the face. The mount may comprises a portion which extends from an interior surface of the side opening and provides a curved surface which engages the nasopharangeal airway. The mount may contact the interior surface of the side opening in at least two different locations. A substantial portion of the mount is formed integrally with the bite block.

What is also described is bite block for use with a nasopharangeal airway, comprising: a main bite block body defining upper and lower outer surfaces adapted to be positioned between a patient's upper and lower teeth respectively and defining an operating lumen for providing instrument access; wherein the bite block includes an outer face adapted to be positioned outside the patient's mouth during use, the outer face defining a pair of side openings; and a mount configured so as to hold a nasopharyngeal airway in one of the side openings such that the nasopharyngeal airway functions as an oral pharyngeal airway. The mount is molded integrally with the bite block. The mount may be disposed within one of the side openings and contacts the interior surface of the side opening in at least two separate locations. The mount may be configured to mate with the flared end of the nasopharyngeal airway in a friction fit or snap fit arrangement. The mount may defines opposing interior surfaces which are curved and tapered in correspondence with the curvature and taper of the proximal end portion of the nasopharangeal airway.

What is also described is a novel suturing device utilizing transverse circular needles, wherein the needles have dual purpose notches which are engaged by a drive shuttle to advance the needles and which are engaged by a biased plunger at the completion of each stroke. These notches may be on the inside surface of the needles. There may be three notches for three strokes per complete revolution. The suturing device may include a confirmation rod which must be withdrawn before the needles can be moved from their initial position. The drive shuttle may be activated by pull wires. The pull wires may be attached to a rotating hub in the handle wherein the hub is driven by a squeeze trigger.

What has been described includes a novel excision device having a mechanism to prevent capture of unwanted tissue in a suction cavity. The mechanism may include a plurality of extendable members disposed on opposing sides of the suction cavity. The extendable members may be contained in recesses when in their non-extended condition. The extendable members may be wires, and the wires may be fixed at one end and extend outwardly under a compressive force.

What has also been described is a novel excision device having a raisable platform in a suction cavity for assisting removal of the excised tissue. The platform may be recessed in the floor prior to being elevated. The platform may be elevated via a plurality of push wires. The platform may have a plurality of openings to accommodate suction holes and/or injections needles in the floor of the suction cavity.

What has also been described is a novel excision device having a lockout mechanism to prevent premature excision of tissue. The lockout mechanism may be used to prevent excision prior to delivery of an injectate into the captured tissue. The lockout may be constructed such that it is automatically released when an injection syringe is attached at a handle mount.

What has also been described is a novel excision device with injection needles which can be extended upwardly from the floor of the cavity and then retracted into the floor after injection.

The novel excision devices described herein may be used to excise surface layers of tissue in the stomach, esophagus, colon, or other parts of GI tract. The may be used alone or in combination with suturing devices, such as any of the suturing devices described herein.

What is also described is a novel suturing device utilizing transverse circular needles, wherein the needles have dual purpose notches which are engaged by a drive shuttle to advance the needles and which are engaged by a biased plunger at the completion of each stroke. These notches may be on the inside surface of the needles. There may be three notches for three strokes per complete revolution. The suturing device may include a confirmation rod which must be withdrawn before the needles can be moved from their initial position. The drive shuttle may be activated by pull wires. The pull wires may be attached to a rotating hub in the handle wherein the hub is driven by a squeeze trigger.

What is also described is a novel suturing device with transverse circular needles driven across a suction cavity in multiple stages by a shuttle, wherein the shuttle is activated by pull wires attached to a hub driven by a squeeze trigger. The squeeze trigger may turn the hub in one direction with the hub designed to snap back under the force of a spring upon completion of each stroke, with multiple strokes forming a complete revolution of the needles. The handle may include a counter to indicate the status of needle activation. The device may include a lock spring/lock rod which engages notches in the needles at completion of each stroke. The squeeze trigger may be constructed such that it can be selectively disengaged from the hub to release tension on the pull wires.

What is also described is a suturing device with transverse circular needles contained in channels defined between adjacent parts of a multi-part capsule, wherein the capsule parts can be separated to release the needles. The capsule parts may be held together by a compressive force, which compressive force may be released at the handle. The compressive force may be exerted by a tie rod. The rod may be threaded and the compressive force released by unscrewing the rod.

What is also described is a suturing device with a plurality of transverse circular needles each with an associated drive shuttle wherein each needle and drive shuttle are contained between a different pair of capsule parts and the capsule parts are configured so as to be spread apart longitudinally.

What is also described is a novel system for performing gastroplasty and GERD procedures comprising a standalone excision device and a standalone suturing device which are each designed to be used without being inserted through a common keeper. One or both may be designed to be inserted over a guidewire. Each may have a handle section, an elongated body, and a distal working portion with a suction cavity. Each may also have an endoscope lumen in their respective elongated body. The endoscope lumen may be designed such that the endoscope enters in the handle and exits proximal to the suction cavity. An endoscope exit hole may be provided in each device by a flexible transition section between a main elongated body and the working portion. The flexible transition section may be of different (more flexible) material than the main elongated body and/or distal working portion.

What is claimed is:

1. A system for performing a gastrointestinal procedure, comprising:
an excision device comprising a first handle section connected to a first working portion via a first elongated flexible shaft, wherein the first working portion comprises a first elongated side disposed suction cavity for capturing tissue and a tissue excision device adapted to traverse the first suction cavity upon activation by an excision actuator in the first handle section so as to excise a layer of tissue captured therein; and
a suturing device comprising a second handle section connected to a second working portion via a second elongated flexible shaft, wherein the second working portion comprises a second side disposed suction cavity and a plurality of needles configured to apply a plurality of transverse sutures to tissue captured in the second suction cavity;
wherein at least one dimension of the first suction cavity is different than a corresponding dimension of the second suction cavity.

2. The system of claim 1 wherein the width of the first suction cavity varies along its length and the width of the second suction cavity is generally constant along its length.

3. The system of claim 2 wherein the width of the first suction cavity is tapered proximally such that a tapered section of exposed tissue is produced.

4. The system of claim 1 wherein the excision device further comprises at least one member which is extendable from a lateral side of the first suction cavity upon activation by an extender actuator in the first handle section so as to prevent the capture of unwanted tissue in the first suction cavity.

5. The system of claim 4 wherein the at least one member comprises a wires which are extendable from a first position in which the wires are disposed in corresponding channels in the outer surface of the working portion.

6. The system of claim 1 wherein a platform is provided in the floor of the first suction cavity which is raisable upon activation by a platform actuator in the first handle section so as to facilitate removal of the excised tissue.

7. The system of claim 1 wherein the plurality of needles are curved needles which are driven in a generally circular path by way of at least one drive shuttle which engages notches on an interior surface of the needles.

8. The system of claim 8 wherein the second working portion comprises a plurality of longitudinally translatable capsule parts wherein the needles are contained in channels defined between adjacent ones of the capsule parts when the parts are together.

9. The system of claim 8 wherein the capsule parts are held together via application of a compressive force and are biased to spread apart when the compressive force is released.

10. An excision device for excising tissue from inside an organ during a minimally invasive procedure comprising:
a handle section connected to a working portion via an elongated flexible shaft, wherein the working portion comprises an elongated side disposed suction cavity for capturing tissue and a tissue excision device adapted to traverse the suction cavity upon activation by an excision actuator in the handle section so as to excise a layer of tissue captured therein;
wherein the working portion further comprises first and second members which are extendable from the lateral sides of the suction cavity upon activation by an extender actuator in the handle section so as to prevent the capture of unwanted tissue in the suction cavity.

11. The excision device of claim 10 wherein the first and second members are extendable by first and second extender actuators respectively.

12. The excision device of claim 11 wherein the members are extendable from a first position in which the members are generally flush with an outer surface of the working portion to a second position in which the members are spaced from the outer surface.

13. The excision device of claim 10 wherein the first and second members comprise wires which bow outwardly from the sides of the suction cavity.

14. The excision device of claim 10 wherein the width of the suction cavity is tapered proximally such that a tapered section of exposed tissue is produced.

15. A suturing device for applying transverse sutures to tissue; comprising:
a handle section connected to working portion via an elongated flexible shaft, wherein the working portion comprises a side disposed suction cavity and a plurality of needles configured to apply a plurality of transverse sutures to tissue captured in the second suction cavity;
wherein the needles are contained in channels defined between adjacent parts of a multi-part capsule.

16. The suturing device of claim 15 wherein the capsule parts are held together via application of a compressive force and are biased to spread apart when the compressive force is released.

17. The suturing device of claim 16 wherein the compressive force is releases at the handle.

18. A suturing device for applying transverse sutures to tissue, comprising:
a handle section connected to working portion via an elongated flexible shaft, wherein the working portion comprises a side disposed suction cavity and a plurality of needles configured to apply a plurality of transverse sutures to tissue captured in the second suction cavity;
wherein the needles are curved needles which are driven in a generally circular path by way of at least one drive shuttle which engages notches on an interior surface of the needles.

19. The combination of a bite block and an airway, wherein the bite block comprises a body defining a main lumen which terminates distally in the oral cavity and a pair of side openings, and wherein the airway is mounted in one of the side openings and extends into the patient's oral cavity substantially past the distal edge of the main lumen.

20. The combination of claim 19 wherein the airway comprises an elongated flexible tube having a flared proximal end.

21. The combination of claim 20 wherein the airway comprises a nasopharyngeal airway.

22. The combination of claim 21 wherein the airway is secured to the bite block via a snap fit or a friction fit mounting arrangement.

23. A system comprising:
a main bite block body defining upper and lower outer surfaces adapted to be positioned between a patient's upper and lower teeth respectively and defining an operating lumen for providing instrument access;
wherein the bite block includes an outer face adapted to be positioned outside the patient's mouth during use, the outer face defining a pair of side openings; and
a nasopharyngeal airway positioned in one of the side openings and extending distally into the patient's oral cavity so as to function as an oral pharyngeal airway.

24. The system of claim 23 wherein the nasopharyngeal airway is secured to the bite block.

25. The system of claim 24 wherein the nasopharyngeal airway is secured to the bite block via a strap or a mount.

26. The system of claim 25 wherein the nasopharyngeal airway is secured to the bite block via a mount which is disposed within the respective side opening of the face.

27. The system of claim 26 wherein the mount comprises a portion which extends from an interior surface of the side opening and provides a curved surface which engages the nasopharangeal airway.

28. The system of claim 26 wherein the mount contacts the interior surface of the side opening in at least two different locations.

29. The system of claim 28 wherein a substantial portion of the mount is formed integrally with the bite block.

30. A bite block for use with a nasopharangeal airway, comprising:
a main bite block body defining upper and lower outer surfaces adapted to be positioned between a patient's upper and lower teeth respectively and defining an operating lumen for providing instrument access;
wherein the bite block includes an outer face adapted to be positioned outside the patient's mouth during use, the outer face defining a pair of side openings; and
a mount configured so as to hold a nasopharyngeal airway in one of the side openings such that the nasopharyngeal airway functions as an oral pharyngeal airway.

31. The bite block of claim 30 wherein the mount is molded integrally with the bite block.

32. The bite block of claim 30 wherein the mount is disposed within one of the side openings and contacts the interior surface of the side opening in at least two separate locations.

33. The bite block of claim 30 wherein the mount is configured to mate with the flared end of the nasopharyngeal airway in a friction fit or snap fit arrangement.
